(19) United States
(12) Patent Application Publication
(10) Pub. No.: US 2015/0139972 A1
(43) Pub. Date: May 21, 2015

(54) Micronutrient Formulation For Concussive Head Injuries

(71) Applicants: Gerald Haase, Greenwood Village, CO (US); Kedar Prasad, San Rafael, CA (US)

(72) Inventors: Gerald Haase, Greenwood Village, CO (US); Kedar Prasad, San Rafael, CA (US)

(21) Appl. No.: 14/544,011

(22) Filed: Nov. 13, 2014

Related U.S. Application Data
(60) Provisional application No. 61/962,802, filed on Nov. 18, 2013.

(51) Int. Cl. A61K 33/30; A61K 31/375; A61K 31/593; A61K 31/122; A23L 2/52; A61K 33/06; A61K 31/4188; A61K 31/525; A61K 31/51; A61K 45/06; A61K 31/355; A61K 31/714

(52) U.S. Cl. CPC A61K 33/30; A61K 31/355; A61K 31/375; A61K 31/593; A61K 31/122; A61K 31/714; A61K 33/06; A61K 31/4188; A61K 31/525; A61K 31/51; A61K 45/06; A23L 2/52; A23V 2002/00
USPC 424/94.1; 514/690

(57) ABSTRACT

A concussive injury micronutrient formulation is provided and the formulation comprises vitamin A, vitamin E, natural mixed carotenoids, vitamin C, vitamin D, coenzyme Q10, alpha-lipoic acid, N-acetyl cysteine, acetyl L-carnitine (fumarate), vitamin B, folic acid, calcium, magnesium, selenium, chromium, biotin, zinc, and omega-3 fatty acids.

### RELATED APPLICATION

This application is related to U.S. Provisional Application Ser. No. 61/962,802, entitled “Micronutrient Formulations for Concussive Brain Injuries” which was filed on Nov. 18, 2013.

### BACKGROUND OF THE INVENTION

### Field of the Invention

The present invention relates to micronutrient formulations for treatment and/or prevention of traumatic brain injuries (TBI) such as concussions.

### SUMMARY OF THE INVENTION

In one embodiment, the present invention provides for a micronutrient formulation comprising vitamin A, vitamin E, natural mixed carotenoids, vitamin C, vitamin D, coenzyme Q10, alpha-lipoic acid, N-acetyl cysteine, acetyl L-carnitine (fumarate), vitamin B, folic acid, calcium, magnesium, selenium, chromium, biotin, zinc, and omega-3 fatty acids wherein said formulation is designed for treatment and prevention of concussions.

In another embodiment, the present invention relates to a micronutrient formulation comprising: vitamin A, vitamin E, vitamin C, vitamin D, coenzyme Q10, N-acetyl cysteine, acetyl L-carnitine, vitamin B, folic acid, calcium, magnesium, selenium, chromium, biotin, zinc, sodium, potassium, carbohydrates wherein said formulation is designed to treat and prevent concussions.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

The specific example below will enable the invention to be better understood. However, they are given merely by way of guidance and do not imply any limitation.

Traumatic brain injury (TBI) occurs when sudden trauma causes damage to the brain. TBI can occur with or without penetrating head injury. TBI without penetrating head injury is known as a concussive brain injury that may be expressed as a mild, moderate or severe form. A concussion occurs when the brain is violently rocked back and forth within the skull following a blow to the head or neck, such as observed in vehicular accidents and contact sports like football, or when the victim is in close proximity to a concussive blast pressure wave, such as in industrial or combat-related circumstances. Because there is no penetration of the skull, concussive brain injury may be difficult to identify after the inciting event. Therefore prevention strategies and standardized treatment guidelines remain elusive. This patent exploits an understanding of the mechanisms of cellular damage, the biological events that impact long-term neurologic outcomes, and the application of antioxidant science to develop a rational strategy for reducing the progression of TBI.

Both experimental and human studies suggest that increased levels of oxidative stress from excessive production of free radicals (1-5), products of uncontrolled acute and chronic inflammation (6-12) and release of a neurotoxic substance, glutamate, in the brain are involved in the progression of damage following concussive injury, and contribute to the initiation and progression of brain damage. These observations are supported by the fact that individual antioxidants (known to neutralize free radicals, reduce inflammation, and prevent the release and toxicity of glutamate), have reduced damage when administered either before or soon after concussion (2, 4, 13, 14). The expression of oncogene proteins, c-myc and c-fos, is elevated in rat brains after concussion (15, 16), and cortical expression of nuclear factor kappa B (NF-kappa B) is elevated in human contused brain (17). The fact that antioxidants and their derivatives reduce expression of c-myc oncogene (18) and activation of NF-kappa B (19), further suggests their value in reducing brain damage following concussive injury. This application presents the scientific rationale and evidence that a specific formulation of multiple micronutrients, including dietary and endogenous antioxidants and glutathione-elevating agents, may prevent and improve standard therapy for TBI, and defines the composition, dosage, and dose schedule that most effectively impacts the acute- and long-term management of concussive brain injury.

### Incidence and Cost of TBI

Annually, about 1.4 million people in the U.S. suffer from TBI, of which 1.1 million are treated and released from an emergency department, 235,000 are hospitalized, and 50,000 die (20). Among children ages 0 to 14 years, concussive brain injuries cause 2685 deaths, 37,000 hospitalizations and 435,000 emergency room visits each year. The CDC Injury Center has estimated that about 3.8 million sports- and recreation-related concussions occur annually in the U.S. In total, at least 5.3 million Americans who suffered from a TBI have long-term or life-long need for assistance to perform the routine activities of daily living. The cost per year per person with mild TBI is about $32,000; with moderate to severe TBI, it is from about $268,000 to more than $408,000. In 2000, the direct medical costs and indirect costs such as lost productivity because of concussive brain injury were estimated to be $60 billion in the U.S. (21). In the military, this is becoming a serious issue as about 30% of all soldiers with combat-related injuries seen at Walter Reed Army Medical Center from 2003 to 2005 sustained a concussive blast. By 2011, about 320,000 soldiers who served in Iraq and Afghanistan suffered a TBI.

### Symptoms and Consequences of Concussive Injury

Symptoms of concussive injury can be mild, moderate or severe, depending upon the extent of damage to the brain. Individuals with a mild TBI may remain conscious or may become unconscious for a few second or minutes, and may exhibit headache, confusion, lightheadedness, dizziness, blurred vision, tinnitus (ringing in the ears), fatigue, alterations in sleeping patterns, mood changes, trouble with memory, concentration, and thinking. Individuals with a moderate or severe TBI can suffer additional symptoms including a headache that gets worse or remains persistent, repeated vomiting or nausea, convulsions or seizures, an inability to awaken from sleep, dilation of one or both pupils of the eyes, slurred speech, weakness or numbness in the extremities, loss of coordination, and increased confusion, restlessness or agitation. This impact on long-term quality of life may affect individuals with TBI for extended periods of time.

Despite evolutionary changes in protective equipment, concussive injuries remain a major health risk for those who engage in potentially violent sports (22, 23) or hazardous occupations, including military combat. Cerebral concussion is a type of TBI that is normally produced by acceleration and deceleration of the head. It can also happen during the rapid displacement and rotation of the cranium after peak head acceleration and momentum transfer by helmet impacts (24). It is characterized by a sudden brief impairment of consciousness, paralysis of reflex activity and loss of memory. Sport-related concussions have been classified into simple and complex concussions (recommendation of the Second International Conference on Concussion in Sport, 2005). Athletes who are slow to recover (i.e., greater than 10 days) are classified having complex concussions. Brain deformation may occur after the primary head acceleration (24). Damage to the mid brain correlated with memory and cognitive problems after concussion. The major depression commonly observed after concussion contributes to impairment of memory, processing speed, verbal memory and executive function (25-27). An early onset of dementia may be initiated by repetitive cerebral concussions in professional football players (28, 29). Balance disorders are also considered one of the major health problems associated with the TBI (29, 30). Current efforts on reducing the impact of concussion have focused on the development of physical protection. Indeed, the introduction of newer football helmets appears to lower the risk of concussion by about 10-20% (30).

Extensive evaluation of the relationship between post-traumatic stress disorder (PTSD) and concussive brain injury has been demonstrated in two recent epidemiologic studies in U.S. soldiers returning from Iraq and Afghanistan (31, 32). Among 2525 soldiers, 4.9% reported injury with loss of consciousness, 10.3% reported injuries with altered mental status, and 17.2% reported other injuries during deployment. Among those who reported loss of consciousness, the incidence of PTSD was about 43.9%. Among those reporting altered mental status, it was 27.3%, and among those reported other injuries, it was 16.2%. In contrast, among those soldiers reporting no injury in combat, the incidence of PTSD was only 9.1%. This patent describes a novel biological protection strategy to reduce the acute and long-term impact of concussion and would be complimentary to existing physical protection. The development of this strategy is based on the biochemical events that initiate damage and that contribute to the progression of damage following concussion.

Biochemical Events Contributing to Damage Progression after TBI

Both animal and human studies show that TBI causes a significant loss of cortical tissue at the site of injury (primary damage). This is followed by secondary damage involving increased oxidative damage, mitochondrial dysfunction, release of predominantly pro-inflammatory cytokines and toxic levels of glutamate leading to cognitive impairment and neurological dysfunction. Therefore, attenuation of these biochemical events may help to reduce the onset and progression of damage.

Increased oxidative stress due to production of excessive amounts of free radicals derived from oxygen and nitrogen occur after concussions (33-36). The extent of oxidative damage appeared to be directly proportion to the severity of TBI (37). In rats, there appears to be a close relationship between the degree of oxidative stress and severity of brain damage following concussive brain injury as evidenced by the high levels of malondialdehyde (lipid peroxidation marker of oxidative damage) and low levels of ascorbate (antioxidant vitamin) (38). TBI induced peroxynitrite-mediated oxidative damage to mitochondrial function that precedes neuronal loss in the brain. The oxidation of nitric oxide (NO) forms peroxynitrite. Animal studies show that TBI increased NO production that impairs mitochondrial function by inhibiting cytochrome oxidase (39). Cytochrome oxidase is a key enzyme needed to generate energy. Thus, the energy level in tissue decreased after concussive injury and this may interfere with repair processes. TBI also increased inducible nitric oxide synthase (iNOS) activity that contributes to neurological deficits by generating excessive amounts of NO (40). In a rat model of traumatic injury (unilateral moderate cortical contusion), increased oxidative damage occur as early as 3 hours following TBI that adversely affects synaptic function and neuronal plasticity, and thereby, enhances cognitive dysfunction (41). In another model of TBI (fluid percussion brain injury in rat), it was observed that levels of oxidative stress increased in the cortex of the brain when measured one and three months following injury.

Human studies also confirm the role of oxidative stress in the progression of TBI. F2-isoprostane is a marker of lipid peroxidation, whereas neuron-specific enolase (NSE) is considered a marker of neuronal damage. The levels of F2-isoprostane and NSE increased in the cerebral spinal fluid (CSF) following concussive brain injury in children and infants (42). The levels of the antioxidants ascorbate and glutathione decreased in the CSF of children and infants following TBI (43). It has also been reported that the levels of beta-amyloid fragment (A beta-42) increased in the CSF of patients after severe concussive brain injury (44). This peptide has been implicated in causing neuronal damage in patients with Alzheimer's disease (45-48).

### Mitochondrial Dysfunction in Concussive Brain Injury

Increased oxidative stress contributes to the mitochondrial dysfunction that plays a central role in causing cognitive impairment and eventually cell death following TBI (49, 50). In a rat model, several mitochondrial proteins involved in bioenergetics were oxidized following injury causing mitochondrial dysfunction (51). In another rat model, it was found that the activity of the mitochondrial enzymes decreased, while acid-base balance was disrupted and levels of oxidative stress increased in the blood following concussions. These changes contribute to the severity of brain injury. Generally, oxidative stress-induced mitochondrial dysfunction is observed one to three hours after TBI, suggesting the importance of early intervention to reduce the oxidative stress (52). There is a direct link between energy metabolism and N-acetylaspartate. In a human clinical study involving 14 patients (6 patients with diffuse brain injury and 8 with focal brain lesions), it was observed that reduction in the brain levels of n-acetylaspartate in the absence of ischemic insult reflected mitochondrial dysfunction (53).

### Increased Inflammation in TBI

Following TBI, brain cells generate excessive amounts of pro-inflammatory cytokines, prostaglandins, reactive oxygen species, complement proteins and adhesion molecules that are highly toxic to neurons (54-58). Evidence of inflammation is also found by the infiltration and accumulation of polymorphonuclear leukocytes. Pro-inflammatory cytokines increased the expression of the enzyme inducible nitric oxide synthase (iNOS) producing excessive amounts of NO that can become oxidized to form peroxynitrite and contributes to the pathogenesis of concussive brain injury (59-61). An inhibitor of iNOS provided neuroprotection against damage produced by peroxynitrite (62). The pro-inflammatory cytokine interleukin-6 (IL-6) is elevated in patients with acute TBI, and a significant relationship exists between the severity of TBI and the IL-6 level (63). In a clinical study involving 75 patients with moderate to severe concussions, the role of cytokines and lipids relative to 30-day mortality was evaluated. The results showed that the levels of cytokines (IL-6 and IL-8) increased and lipid decreased in all patients, especially non-survivors, compared to those in the healthy control group (64). In addition, severe TBI in infants and children (N=36) increased the levels of pro-inflammatory cytokines (IL-1beta, IL-6 and IL-12p70) and anti-inflammatory cytokines (IL-10) and chemokines (IL-8 and MIP-1alpha) compared to controls.

In animal models, the levels of inflammation markers such as iNOS and cyclooxygenase 2 activity, and markers of oxidative stress (loss of glutathione and oxidized: reduced glutathione ratio, 3-nitrotyrosine, and 4-hydroxynonenal) increased after concussions (65). Motor performance and spatial memory acquisition were improved in genetically protected mice compared to control mice after subjecting them to a brain impact model of TBI (66). The role of pro-inflammatory cytokines in the progression of damage following concussive injury is further supported by the fact that inhibitors of these cytokines improved neuronal loss and cognitive dysfunction.

### Increased Glutamate Release in TBI

The excitatory amino acids (glutamate and aspartate) play a significant role in the progression of injury following concussions (67). Excessive amounts of glutamate in the extracellular space may cause swelling, edema and eventually cell death. In a clinical study involving 80 patients with severe head injury, it was observed that the levels of excitatory amino acids increased and enhanced neuronal damage (68). In patients with focal and diffuse brain injury, the levels of glutamate were elevated in both cerebrospinal fluid and extracellular space (69). In another clinical study, it was found that patients who died of their head injury had higher levels of dialysate glutamate and aspartate compared to those who recovered (67). It was also observed that the levels of adenosine and glutamate were elevated in the ventricular CSF in 27 children with severe TBI compared to 21 children without concussive brain injury (70). The involvement of glutamate in the progression of damage following concussion is further suggested by the fact that administration of antagonists to glutamate release improved motor function and cognitive dysfunction in animal models of TBI (71, 72).

Treatment of Concussive Brain Injury with Antioxidants

Since increased oxidative stress, chronic inflammation and glutamate release are involved in the development and progression of neurological deficits, and since antioxidants are known to reduce these factors, a novel biological protection strategy of daily supplementation with appropriate types of multiple micronutrients including dietary and endogenous antioxidants can be expected to reduce the risk of neurological dysfunction.

Resveratrol, a phenolic antioxidant, administered immediately after TBI reduced oxidative damage and lesion volume in rats (73, 74). Edaravone, an FDA approved drug, reduced oxidative damage by neutralizing free radicals after concussive injury in humans (75). Superoxide dismutase improved TBI-induced mitochondrial dysfunction in mice (76). Treatment with alpha-lipoic acid reduced markers of pro-inflammatory cytokines and oxidative stress, and improved histological changes in the brain, preserved blood-brain-barrier permeability and reduced edema following TBI in animals (77). Administration of N-acetylcysteine provided neuroprotection in animal models following concussion by reducing markers of pro-inflammatory cytokines and adhesion molecules (78). Melatonin, a pineal hormone exhibiting antioxidant activity, protected against TBI-induced damage (79). Dietary supplementation with vitamin E or curcumin protected the brain against damage after concussive injury by reducing the biochemical changes involved in synaptic plasticity and cognitive function (14, 80, 81). Vitamin E also inhibited the release and toxicity of glutamate (82, 83). Dietary supplementation of omega-3 fatty acids in an animal model protected against TBI-induced reduced synaptic plasticity and cognitive impairment (84). In addition to the data relative to oxidative damage, there is substantial evidence that dietary and endogenous antioxidants and antioxidants derived from herbs and fruits and vegetables inhibit inflammation (85-97). Increased pro-inflammatory stimuli and oxidative stress cause brain tissue to release excessive amounts of glutamate after TBI, which contributes to loss of neurons (98). Release of glutamate was blocked by vitamin E (98). Both vitamin E (83) and coenzyme Q10 (82) also protect against glutamate-induced neurotoxicity in cell culture models.

### Rationale for Employing Multiple Micronutrients in TBI

While laboratory studies in animal models show that supplementation with a single micronutrient such as an antioxidant may protect the brain against TBI-induced biochemical and structural damage, similar clinical studies have produced inconsistent results. It is well established that the internal oxidative environment in these populations is high, and that an individual antioxidant when oxidized acts as a pro-oxidant. Therefore, administration of a single antioxidant under high risk conditions may produce pro-oxidant rather than antioxidant effects. Because increased levels of oxidative stress and chronic inflammation, as well as enhanced release of glutamate follow TBI, oral supplementation with appropriate multiple micronutrients including dietary and endogenous antioxidants as an adjunct to standard therapy can be expected to reduce the risk of late adverse effects on brain function. In addition, when administered in a concussion-prone environment, but before the event occurs, they may prevent or reduce the level of initial damage.

For the purpose of uniqueness in this patent application, specific comprehensive combinations of antioxidants and glutathione-elevating agents is mandatory because their mechanisms of action and distribution at cellular and organ levels differ, their cellular and organ environments (oxygenation, aqueous and lipid components) differ, and their affinity for various types of free radicals differs. For example, beta-carotene (BC) is more effective in quenching oxygen radicals than most other antioxidants (99). BC can perform certain biological functions that cannot be produced by its metabolite vitamin A, and vice versa (100, 101). It has been reported that BC treatment enhances the expression of the connexin gene which codes for a gap junction protein in mammalian fibroblasts in culture, whereas vitamin A treatment does not produce such an effect (101). Vitamin A can induce differentiation in certain normal and cancer cells, whereas BC and other carotenoids do not (102, 103). Thus, BC and vitamin A have, in part, different biological functions.

The gradient of oxygen pressure varies within cells. Some antioxidants, such as vitamin E, are more effective as quenchers of free radicals in reduced oxygen pressure, whereas BC and vitamin A are more effective in higher atmospheric pressures (104). Vitamin C is necessary to protect cellular components in aqueous environments, whereas carotenoids and vitamins A and E protect cellular components in lipid environments. Vitamin C also plays an important role in maintaining cellular levels of vitamin E by recycling vitamin E radical (oxidized) to the reduced (antioxidant) form (105). Also, oxidative DNA damage produced by high levels of vitamin C could be protected by vitamin E. Oxidized forms of vitamin C and vitamin E can also act as radicals; therefore excessive amounts of any one of these forms, when used as a single agent, could be harmful over a long period of time.

The form of vitamin E used is also important in any clinical trial. It has been established that d-alpha-tocopheryl succinate (alpha-TS) is the most effective form of vitamin both in vitro and in vivo (106, 107). This form of vitamin E is more soluble than alpha-tocopherol and enters cells more readily, and, therefore, is expected to cross the blood-brain barrier more efficiently in TBI.

Glutathione is one of the body's most important antioxidants. However, oral supplementation of this substance failed to significantly increase plasma levels of glutathione in human subjects (108) suggesting that this tripeptide is completely hydrolyzed in the gastrointestinal tract. N-acetylcysteine and alpha-lipoic acid increase the cellular levels of glutathione by different mechanisms and can be effectively combined in a multiple micronutrient preparation. Selenium is a co-factor of glutathione peroxidase that also increases the intracellular level of glutathione. In addition, R-alpha-lipoic acid and acetyl-L-carnitine together promoted mitochondrial biogenesis whereas no effect was observed when these antioxidants were used individually (109).

Other endogenous antioxidants, such as coenzyme Q₁₀, also have potential value in prevention and adjunctive treatment of TBI. Since mitochondrial dysfunction occurs in patients with concussive injury and since coenzyme Q₁₀ is needed for the generation of ATP by mitochondria, this antioxidant is essential to improve the mitochondrial function. Ubiquinol (coenzyme Q₁₀) scavenges peroxy radicals faster than alpha-tocopherol (110) and like vitamin C, can regenerate vitamin E in a redox cycle (111). Coenzyme Q₁₀ administration has also been shown to improve clinical symptoms in patients with mitochondrial encephalomyopathies (112).

Antioxidant micronutrients can reduce the risk of neurological dysfunction in other conditions such as Alzheimer's and Parkinson's disease, and this neuroprotective value is directly relevant to concussive brain injury and the common late adverse effects. Beta-amyloid fragments that are associated with neurodegeneration in Alzheimer's disease mediate their action by free radicals (113). This is supported by the fact that vitamin E protects neuronal cells in culture against beta-amyloid-induced toxicity (114). Vitamin E at a dose of 2,000 IU per day can produce beneficial effects in patients with Alzheimer's disease (115). Patients consuming antioxidants also showed reduced risk of vascular dementia and slower decline of cognitive function in cases of dementia and Alzheimer's disease (116). Prostaglandin E2, a product of inflammatory reactions, is very toxic to mature neurons, and a mixture of antioxidants reduces this toxicity (117). Furthermore, glutathione deficiency has been consistently found in autopsied brain samples from patients with neurological conditions such as Alzheimer's disease (118) and Parkinson disease (119, 120), again demonstrating the relevance of employing micronutrients that increase glutathione for TBI.

### Micronutrient Formulations for Concussive Brain Injury

The formulations for concussive brain injury can be placed in the following forms capsules, pills, beverages, powders, chewables (e.g. gummies), dissolvables (e.g. oral wafers and disks), solids (e.g. bars), functional foods (e.g. yogurts and creams), sprays, inhaled aerosols, suspensions, liposomes, extended release technologies, and other absorbable forms such as skin patches and injectables.

These comprehensive formulations are intended for individuals who have already suffered initial or repeated TBI from contact sports, accidents, dangerous occupational environments or military combat, or for those who may likely suffer such an event because of exposure to these high risk circumstances. The cognitive dysfunction and other neurological abnormalities may be noted soon after concussions or may not be evident until several years later.

### Examples of Micronutrient Formulations

### Example 1

A formulation for “youth”, for example ages 9-13 years, is provided in a bottled form (two capsules taken twice per day), liquid form (one packet dissolved in liquid twice per day), beverage bottle (8-12 ounces) or bar form (one bar consumed twice per day) and is recommended (but not required) to be taken year round by young athletes.

One serving of the daily consumption (two servings per day) is comprised of: vitamin A (retinyl palmitate, fish oil, or natural mixed carotenoids) 1500 IU, vitamin E (both d-alpha-tocopherol 50 IU and d-alpha tocopheryl succinate 50 IU, vegetable products, natural tocopheryl, or wheat germ products),

vitamin C (calcium ascorbate, citrus, rose, or berry products) 250 mg,
vitamin D (cholecalciferol-D3, or fish oils) 200 IU,
coenzyme Q10 (ubiquinone) 45 mg,
R-alpha-lipoic acid (R+ or R−) 15 mg,
N-acetyl cysteine 25 mg,
acetyl L-carnitine (fumarate) 50 mg,
vitamin B1-(thiamine mononitrate or yeast sources) 2 mg,
vitamin B2-(riboflavin or yeast sources) 2.5 mg,
vitamin B3-(niacinamide or yeast sources) 5 mg,
vitamin B6-(pyridoxine hydrochloride or yeast sources) 2.5 mg,
folic acid (folate, yeast source, or liver sources) 200 mcg,
vitamin B12-(cyanocobalamin or yeast source) 5 mcg,
biotin (d-biotin or liver sources) 100 mcg,
vitamin B-5 (pantothenic acid, D-calcium pantothenate, pantothenate, yeast sources, rice bran, or
liver sources) 5 mg,
calcium (citrate, ascorbate, plant based sources, or lactate) 125 mg,
magnesium (citrate, lactate, or natural forms) 62.5 mg,
selenium (seleno-L-methionine or natural forms) 50 mcg,
zinc (glycinate or natural forms) 7.5 mg, and
chromium (picolinate or natural forms) 25 mcg.

### Example 2

A formulation for “adults”, for examples ages 14 years and older, is provided in a bottled form (three capsules plus one fatty acid gel-cap taken twice per day), liquid form (one packet dissolved in liquid twice per day), beverage bottle (8-32 ounces), or bar form (one bar consumed twice per day) and is recommended (but not required) to be taken year round by those engaging in contact sports, working in hazardous occupations, or those in the active military.

The daily consumption is comprised of:

vitamin A (retinyl palmitate, fish oil, or natural mixed carotenoids) 3000 IU,
vitamin E (both d-alpha-tocopherol 100 IU and d-alpha tocopheryl succinate 300 IU, vegetable products, natural tocopheryl, or wheat germ products),
vitamin C (calcium ascorbate, citrus, rose, or berry products) 1000 mg,
vitamin D (cholecalciferol-D3 or fish oils) 800 IU,
coenzyme Q10 (ubiquinone) 120 mg,
alpha-lipoic acid (R+ or R−) 90 mg,
N-acetyl cysteine 200 mg,
acetyl L-carnitine (fumarate) 150 mg,
vitamin B1 (thiamine mononitrate, or yeast sources) 4 mg,
vitamin B2 (riboflavin, or yeast sources) 5 mg,
vitamin B3 (niacinamide or yeast sources) 20 mg,
vitamin B6 (pyridoxine hydrochloride or yeast sources) 5 mg,
folic acid (folate, yeast source, or liver source) 800 mcg,
vitamin B12 (cyanocobalamin or yeast source) 10 mcg,
biotin (d-biotin or liver sources) 200 mcg,
pantothenic acid (D-calcium pantothenate, yeast, rice bran, or liver sources) 10 mg,
calcium (citrate, ascorbate, plant based sources or lactate) 250 mg,
magnesium (citrate, lactate, or natural forms) 125 mg,
selenium (seleno-L-methionine, or natural forms) 100 mcg,
zinc (glycinate or natural forms) 15 mg,
chromium (picolinate or natural forms) 50 mcg, and
omega-3 fatty acids (EPA:DHA, 3:2) 2000 mg.

### Example 3

A “booster” formulation, for ages 14 years and older, is provided in a package form (one capsule plus one fatty acid gel-cap), liquid form (one packet dissolved in liquid), beverage bottle (2-8 ounces) or bar form (one bar) and is intended to be taken once as a separate “neuroprotective” package one to two hours prior to engaging in potentially violent contact sports, undertaking an occupationally-related hazardous activity where the risk of concussive injury is increased, or, for active military personnel, entering a combat or hazardous duty zone.

The preparation is comprised of:

vitamin E (d-alpha tocopheryl succinate, vegetable products, natural tocopheryl, or wheat germ products) 50 IU,
vitamin C (calcium ascorbate, citrus, rose, or berry products) 200 mg,
coenzyme Q10 (ubiquinone) 25 mg,
N-acetyl cysteine 100 mg,
acetyl L-carnitine (fumarate) 100 mg, and
omega-3 fatty acids (EPA:DHA, 3:2) 1000 mg.

### Example 4

A “protection” formulation, ages 8 years older, is provided in a package form (one capsule plus one fatty acid gel-cap taken twice per day), liquid form (one packet dissolved in liquid twice per day), or bar form (one bar consumed twice per day) and is intended to be taken for the total number of days as directed by health care professional for those who have sustained a concussive brain injury.

The preparation is comprised of:

vitamin E (d-alpha tocopheryl succinate, vegetable products, natural tocopheryl, or wheat germ products) 50 IU,
vitamin C (calcium ascorbate, citrus, rose, or berry products) 100 mg,
coenzyme Q10 (ubiquinone) 45 mg,
N-acetyl cysteine 50 mg,
acetyl L-carnitine (fumarate) 50 mg, and
omega-3 fatty acids (EPA:DHA, 3:2) 500 mg.

Doses in the above examples are recommended but can be flexible. Doses for example formulations 1, 2, & 4 should be taken at least once per day. Example formulations are target doses, but doses may vary within above ranges.

The micronutrient supplements should be taken orally and divided into two doses, half in the morning and the other half in the evening preferably with meals. This is because the biological half-lives of micronutrients (water-soluble and fat-soluble) are highly variable which can create marked fluctuations in tissue levels, differences in gene profile expression and cellular stress. This is also important to maintain relatively consistent levels of micronutrients in the brain.

No iron, copper or manganese would be included because these trace minerals are known to interact with vitamin C to produce free radicals. These minerals are also absorbed more readily from the intestinal tract in the presence of antioxidants, and this could result in potentially harmful increased body stores of unbound minerals. Increased iron stores have been linked to a higher risk of several chronic diseases (121).

### Evidence of Effectiveness of Micronutrient Formulations

### 1) Oxidative Damage Impact:

Prospective, randomized, double-blind, placebo-controlled trial in 34 U.S. Marine Corps 1^{st} Tank volunteers subjected to cold, high altitude and exertion stress at the Mountain Warfare Training Center, 29 Palms, Calif. (122).

The study group consumed the PMC formulation and the control group received a placebo supplement for duration of the 12 week training course. Serial urine samples were taken from all participants and analyzed before and after supplementation for sensitive markers of oxidative damage (8-hydroxyguanosine). Safety was assessed by field reports of adverse reactions.

In the placebo group, 42% of subjects had low levels of the biological marker before supplementation, and the remaining 58% exhibited high levels of oxidative damage reflecting the extreme conditions. After consuming the placebo, only 25% of the subjects still had low levels of oxidative damage and 75% had high levels, demonstrating 17% deterioration in oxidative status.

Conversely, in the antioxidant treated group, 30% of the subjects had low levels of 8-hydroxyguanosine before supplementation and 70% exhibited high levels of the marker. After receiving the PMC formulation, 71% of the subjects showed low oxidative damage levels and only the remaining 29% had high levels, demonstrating 41% improvement in oxidative status (table). This documented that the formulation not only prevented more oxidative stress from occurring during extreme exercise but, in fact, reduced the oxidative damage that was already present. This significant recovery in the treatment group showed a highly desirable impact during military training and translates directly to the exertion and stress experienced in elite or recreational sports, and hazardous occupations.

In addition, the changes in plasma levels of antioxidant micronutrients were also documented. The placebo group showed no difference in the levels of alpha-tocopherol (vitamin E) in the blood, before (4.8 ug·ml) or after (4.6 ug/ml) supplementation. However, the average value almost doubled (4.1 ug/ml to 7.6 ug/ml) in the antioxidant treated group, documenting that the PMC formulation is absorbed well even in the face of extreme conditions and intense exercise. There were no reported or observed adverse effects from consuming the formulation.

TABLE 1

| | | | Military Antioxidant | |
|---|---|---|---|---|
| | Placebo | | Formulation | |
| | | Post- | | Post- |
| | Pre-treatment | treatment | Pre-treatment | treatment |
| | N = 12 | N = 12 | N = 17 | N = 14 |
| | (% of total subjects) | | (% of total subjects) | |
| Low value | 42 | 25 | 30 | 71 |
| High value | 58 | 75 | 70 | 29 |

The 8-hydroxuguanosine level (μg/mg creatine) up to 2.0 was considered low value (low oxidative damage) and 2.1 to 3.0 as high value (high oxidative damage).). The number of subjects with high oxidative damage increased after placebo consumption. However, the number of subjects with high oxidative damage decreased after antioxidant treatment. N refers to the number of subjects in each group.

2) Beneficial Impact after Concussive Brain Injury:

Prospective, randomized, double-blind clinical trial in 42 U.S. Marine Corps personnel suffering TBI from moderate concussive brain injury (blasts) after returning from war in Iraq (123). All patients had received their injury 3-20 weeks prior to study entry. The control group received standard rehabilitation (steroids, physical therapy, vestibular rehabilitation and supportive care) for 12 weeks. The study group received standard care plus the formulation (two capsules by mouth twice per day) for the same time period.

All patients were evaluated by the same outcome measures that included the Sensory Organization Test (SOT) by Computerized Dynamic Posturography (CDP), Dynamic Gait Index (DGI), the Activities Balance Confidence (ABC) scale, Dizziness Handicap Index (DHI), Vestibular Disorders Activities of Daily Living (VADL) score, and the Balance Scoring System (BESS) test. The therapist who performed and graded the testing was blinded as to whether or not the patient was in the control group or receiving antioxidant therapy. The pre-trial test scores did not differ significantly between the two groups on any of the tests.

The study group receiving the micronutrient formulation demonstrated more rapid and complete recovery than did the control group even though the formulation was not consumed until well after the concussions were suffered. Postural stability, dynamic gait index, and dizziness handicap scores were already significantly different after only 4 weeks, and this improvement grew in significance by the end of 12 weeks. The SOT score by CDP was 78 for the antioxidant group as compared to 63 for the control group (P<0.05).

The improvement noted in the micronutrient group on the other tests also trended to a greater degree than that of the control group. Questionnaires were also administered regarding energy levels, exercise, and overall cognitive issues by an investigator who did not know to which group the patient belonged. The study group demonstrated a significant increase in energy level, exercise tolerance and cognitive ability at every weekly time point. There were no adverse effects from the PMC formulation.

### 3) Prevention of Neurological Injury:

This was a laboratory study of Parkinson's disease in a validated rodent model (124). The agent, MPTP (1-methyl-4-phenyl-1,2,3,6-tetrahydropyridine), is a contaminant in certain recreational drugs and can induce Parkinson's disease in humans. This substance can also be administered intraperitoneally in rats and induces severe Parkinsonian symptoms allowing this model to be used to understand the mechanisms of the disease.

The PMC formulation was fed to the study group prior to administration of MPTP and provided a significant degree of neuroprotection compared to the control group. The supplemented rodents demonstrated an 86% reduction in the development of Parkinson's disease compared to the rats that received only standard diets.

### 4) Prevention of Concussion-Related Inflammation

This was a laboratory study of concussive blast overpressure in a validated rodent model (125). The study formulation was administered orally to rats for 7 days before exposure to a whole-body concussive blast dose of 150 kilopascals. This is generally a lethal overpressure dose in rodents, and represents a potentially concussion-producing blast in humans, both of which would cause a significant rise in markers of severe inflammation in the bloodstream.

The supplementation demonstrated a dramatic protective effect in that it completely prevented the rise of inducible nitric oxide synthase, a critical enzymatic marker of the inflammatory cascade in the blood of the animals. In addition, the supplementation did not adversely affect certain enzymatic markers of oxidative damage such as hemeoxygenase-1 and superoxide dismutase.

The formulations in this application support tissue repair and recovery during exertion and extreme conditions as related to oxidative damage and inflammation. The validating studies, now published in the peer-reviewed clinical medical literature (126), show beneficial impact on concussive events, both preventatively before an event occurs, and supportively after an event is suffered.

In addition to the risk of TBI, high intensity activity in sports, occupational or military environments is also associated with enormous production of excess free radicals and results in measurable oxidative damage to the body's cells and tissues. This type of effort also causes significant inflammatory responses in the body and is known to suppress normal immune function. Specific formulations based on biochemical mechanisms in concussive brain injury and exploiting the known antioxidant science have been developed to addresses the risks from this spectrum of violent activity.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A micronutrient formulation for treating and preventing concussion in human beings comprising:
vitamin A, vitamin E, natural mixed carotenoids, vitamin C, vitamin D, coenzyme Q10, alpha-lipoic acid, N-acetyl cysteine, acetyl L-carnitine (fumarate), vitamin B, folic acid, calcium, magnesium, selenium, chromium, biotin, zinc, and omega-3 fatty acids wherein said formulation is designed for treatment and prevention of concussions.

2. The formulation of claim 1, is to be taken orally.

3. The formulation of claim 1, should be taken by humans ages five and up.

4. The formulation of claim 1, where the composition should be taken twice a day.

5. The formulation of claim 1 wherein Vitamin A is selected from a group comprising retinyl palmitate, fish oil, natural mixed carotenoids, and mixtures thereof.

6. The formulation of claim 1, wherein Vitamin E is selected from a group comprising d-alpha-tocopherol, d-alpha tocopheryl succinate, vegetable products, natural tocopheryl, or wheat germ products and mixtures thereof.

7. The formulation of claim 1, wherein Natural Mixed Carotenoids selected from a group comprising natural sea forms, natural plant forms, and mixtures thereof.

8. The formulation of claim 1, wherein Vitamin C is selected from a group of calcium ascorbate, citrus, rose, berries and mixtures thereof.

9. The formulation of claim 1, wherein Vitamin D is selected from a group comprising cholecalciferol-D3, fish oils and mixtures thereof.

10. The formulation of claim 1, wherein alpha lipoic acid selected from a group comprising alpha-lipoic R+, alpha lipoic R−, and mixtures thereof.

11. The formulation of claim 1, where Vitamin B is selected from a group comprising vitamin B1 (thiamine mononitrate), vitamin B2 (riboflavin), vitamin B3 (niacinamide), vitamin B5 (pantothenic acid), vitamin B5 (pantothenate), vitamin B6 (pyridoxine hydrochloride), vitamin B12 (cyanocobalamin), yeast sources, rice bran, liver sources, and mixtures thereof.

12. The formulation of claim 1, where folic acid is selected from a group comprising folate, yeast source, liver source, and mixtures thereof.

13. The formulation of claim 1, where calcium is selected from a group comprising citrate and ascorbate, plant based source, lactate, and mixtures thereof.

14. The formulation of claim 1, where magnesium is selected from a group comprising citrate, lactate, natural form magnesium and mixtures thereof.

15. The formulation of claim 1, where selenium is selected from a group comprising seleno-L-methionine, natural form selenium and mixtures thereof.

16. The formulation of claim 1, where chromium is selected from a group comprising picolinate, natural form chromium, and mixtures thereof.

17. The formulation of claim 1, where biotin is selected from a group comprising d-biotin, liver source and mixtures thereof.

18. The formulation of claim 1, where zinc is selected from a group comprising glycinate, natural form zinc, and mixtures thereof.

19. The formulation of claim 1, where said formulation comprises: vitamin A from about 300IU to about 5,000 IU, vitamin E from about 15 IU to about 800 IU, natural mixed carotenoids from about 1 mg to about 60 mg, vitamin C from about 100 mg to about 2,000 mg, vitamin D from about 100 IU to about 2,000 IU, coenzyme Q10 from about 1 mg to about 150 mg, alpha-lipoic acid from about 1 mg to about 100 mg, N-acetyl cysteine from about 10 mg to about 500 mg, acetyl L-carnitine (fumarate) from about 25 mg to about 200 mg, vitamin B from about 0.001 mg to about 300 mg, folic acid from about 100 mcg to about 800 mcg, calcium from about 25 mg to about 300 mg, magnesium from about 12.5 mg to about 300 mg, selenium from about 10 to about 100 mcg, chromium from about 5 to about 50 mcg, biotin from about 25 mcg to about 250 mcg, zinc from about 0.5 mg to about 20 mg, omega-3 fatty acids from about 250 mg to 3,000 mg, and mixtures thereof.

20. The formulation of claim 1 having a “booster” formulation, comprising vitamin E, vitamin C, coenzyme Q10, N-acetyl cysteine, and acetyl L-carnitine (fumarate).

21. The formulation of claim 20 comprising omega-3 fatty acids.

21. The formulation of claim 1 having a “protection” formulation comprising: vitamin E, vitamin C, N-acetyl cysteine, acetyl L-carnitine (fumarate), coenzyme Q10, and omega-3 fatty acids.

22. A micronutrient formulation for treating and preventing concussion in human beings comprising: vitamin A, vitamin E, vitamin C, vitamin D, coenzyme Q10, N-acetyl cysteine, acetyl L-carnitine, vitamin B, folic acid, calcium, magnesium, selenium, chromium, biotin, zinc, sodium, potassium, carbohydrates wherein said formulation is designed to treat and prevent concussions.

23. The formulation of claim 22 is to be consumed orally.

24. The formulation of claim 22 is to be consumed in a liquid form.

25. The formulation of claim 22 comprising vitamin A from about 300IU to about 5,000 IU, vitamin E from about 15 IU to about 800 IU, vitamin C from about 100 mg to about 2,000 mg, vitamin D from about 100 IU to about 2,000 IU, coenzyme Q10 from about 1 mg to about 150 mg, N-acetyl cysteine from about 10 mg to about 500 mg, acetyl L-carnitine (fumarate) from about 25 mg to about 200 mg, vitamin B from about 0.001 mg to about 300 mg, folic acid from about 100 mcg to about 800 mcg, calcium from about 25 mg to about 300 mg, magnesium from about 12.5 mg to about 300 mg, selenium from about 10 to about 100 mcg, chromium from about 5 to about 50 mcg, biotin from about 25 mcg to about 250 mcg, zinc from about 0.5 mg to about 20 mg, sodium from about 30 mg to about 200 mg, potassium from about to 5 mg to about 75 mg, carbohydrates about 5 grams to about 100 grams and mixtures thereof.

26. A micronutrient formulation for a beverage comprising: carbohydrate, vitamin A, vitamin C, vitamin D-3, Vitamin E, vitamin B-1, vitamin B-2, vitamin B-3, Vitamin B-6, folate, Vitamin B-12, biotin, Vitamin B-5, calcium, magnesium, zinc, selenium, chromium, sodium, potassium, n-acetyl cystine, coenzyme Q10, L-Carnitine and mixtures thereof.

27. Formulation of claim 26 comprising water, sugar, citric acid, natural flavors, fruit juice, vegetable juice, sodium citrate, and potassium citrate.
